(19) United States
(12) Patent Application Publication
(10) Pub. No.: US 2025/0082727 A1
(43) Pub. Date: Mar. 13, 2025

(54) CXCL14-BASED COMPOSITIONS AND METHOD FOR ENHANCING CARTILAGE REGENERATION

(71) Applicant: FAR EASTERN MEMORIAL HOSPITAL, New Taipei City (TW)

(72) Inventors: Hsiu-Jung LIAO, New Taipei City (TW); Chih-Hung CHANG, New Taipei City (TW); Yi-Shan SHEN, New Taipei City (TW); Chi-Ying HUANG, Taipei City (TW)

(73) Assignee: FAR EASTERN MEMORIAL HOSPITAL, New Taipei City (TW)

(21) Appl. No.: 18/827,250

(22) Filed: Sep. 6, 2024

Related U.S. Application Data
(60) Provisional application No. 63/581,085, filed on Sep. 7, 2023.

(51) Int. Cl. A61K 38/19; A61K 9/50; A61K 45/06; A61P 19/02

(52) U.S. Cl. CPC A61K 38/195; A61K 9/5068; A61K 45/06; A61P 19/02

(57) ABSTRACT

The present invention provides a method and a pharmaceutical composition for cartilage regeneration or treatment of a cartilage defect disease, e.g., degenerative osteoarthritis, which comprises CXCL14 protein, or the peptide fragment thereof. The method and pharmaceutical composition could further comprise stem cell-derived extracellular vesicles.

### CROSS REFERENCE

This application claims priority to U.S. Provisional Patent Appl. No. 63/581,085 filed on Sep. 7, 2023, the contents of which is incorporated by reference in its entirety

### FIELD OF THE INVENTION

The invention relates to a method and pharmaceutical composition of CXCL14 protein and the peptide fragment thereof for enhancing cartilage regeneration.

### REFERENCE TO ELECTRONIC SEQUENCE LISTING

The application contains a Sequence Listing which has been submitted electronically in .XML format and is hereby incorporated by reference in its entirety. Said .XML copy, created on Sep. 6, 2024, is named “2024-9-6-SeqListing-5992-0462PUS2” and is 3,040 bytes in size. The sequence listing contained in this .XML file is part of the specification and is hereby incorporated by reference herein in its entirety.

### BACKGROUND

Cartilage is a tissue composed of extracellular matrix and cartilage cells as single cells, which has neither no blood vessels nor nerve. Therefore, it is difficult to heal the cartilage tissue when it is damaged. Further, a cartilage cell is surrounded by hard extracellular matrix, and thus it is difficult to regenerate cartilage once damaged or degenerated.

For treatments of cartilage damage or degeneration, some drugs such as analgesics, steroids, or non-steroidal anti-inflammatory drugs may be used clinically. However, the drug treatment only has an effect of relieving the pain or the inflammatory reaction itself non-specifically. Cartilage protectants such as hyaluronic acid, glucosamine, or chondroitin may also be used to treat damaged cartilage tissue. However, the cartilage protectants temporarily protect the joints by merely nourishing the cartilage cells or alleviating the shocks. In addition, surgical procedures such as arthroscopic surgery, proximal tibial osteotomy, total knee arthroplasty, or bone-cartilage tissue grafting may be used. Various orthopedic surgical procedures are performed, and representative methods therefor include bone marrow stimulation and osteochondral graft. The bone marrow stimulation is an alternative method with stem cells, for example those derived from bone marrow, but it is regenerated as fibrocartilage rather than hyaline cartilage after surgery. The osteochondral graft is a treating method of collecting osteo-cartilage connective tissue at a site that receives less weight in the patient's own cartilage tissue and then transplanting it to the cartilage damage site. However, it cannot be used for a large damage site. In addition, these therapies cannot completely prevent progressive joint destruction and cure degenerative osteoarthritis (OA).

Therefore, it is still desirable to develop a new approach for cartilage regeneration.

### SUMMARY OF THE INVENTION

It is unexpectedly found in the present invention that the CXCL14 gene is highly expressed in human bone marrow cells during chondrogenesis, which can enhance chondrogenesis. Accordingly, the present invention provides a new solution on cartilage regeneration using the CXCL14 protein and the peptide fragment thereof.

In one aspect, the present invention provides a use of CXCL14 protein or peptide fragment thereof in manufacturing a medicament for treating a cartilage defect disease in a subject.

In one further aspect, the present invention provides a method for treating a cartilage defect disease in a subject in need thereof, comprising administering a pharmaceutical composition comprising a therapeutically effective amount of CXCL14 protein or peptide fragment thereof, in association with a pharmaceutically acceptable carrier.

In a yet aspect, the present invention provides a pharmaceutical composition for treating the cartilage defect disease, comprising a therapeutically an effective amount of CXCL14 protein or peptide fragment thereof, in association with a pharmaceutically acceptable carrier.

In one embodiment of the present invention, the medicament further comprises stem cell-derived extracellular vesicles (EV).

In one example of the present invention, the EV contains the CXCL14protein or the peptide fragment thereof.

In some examples of the present invention, the CXCL14 protein is selected from the group consisting of:
(1) a protein comprising an amino acid sequence of SEQ ID NO: 1;
(2) a protein comprising a sequence with at least 80% identity to the amino acid sequence of SEQ ID NO: 1;
(3) a protein comprising a sequence with at least 95% identity to the amino acid sequence of SEQ ID NO: 1; and
(4) a protein comprising a sequence with at least 98% identity to the amino acid sequence of SEQ ID NO: 1.

In some examples of the present invention, the CXCL14 protein is selected from the group consisting of:
(1) a peptide fragment comprising a sequence of SEQ ID NO: 2
(2) a peptide fragment comprising a sequence with at least 80% identity to the amino acid sequence of SEQ ID NO: 2;
(3) a peptide fragment comprising a sequence with at least 95% identity to the amino acid sequence of SEQ ID NO: 2; and
(4) a peptide fragment comprising a sequence with at least 98% identity to the amino acid sequence of SEQ ID NO: 2.

In a specific example of the present invention, the cartilage defect disease is degenerative osteoarthritis (OA).

In a specific example of the present invention, the CXCL14 protein is an amino acid sequence set forth in SEQ ID NO: 1.

In a specific example of the present invention, the peptide fragment thereof is an amino acid sequence set forth in SEQ ID NO: 2.

In a specific example of the present invention, the stem cell-derived extracellular vesicle (EV) is derived from mesenchymal stem cell (MSC) or adipose-derived stem cell (ADSC).

In a specific example of the present invention, the CXCL14 protein, the peptide fragment thereof, or the stem cell-derived extracellular vesicle (EV) is administered to the cartilage damage of the subject via an injection or a surgery.

It is to be understood that both the foregoing general description and the following detailed description are exemplary and explanatory only and are not restrictive of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing description and the following detailed description of the invention will be better understood when reading in conjunction with the accompanying drawings. For the purpose of illustrating the present invention, currently preferred embodiments are shown in the drawings.

FIGS. 1A-1D show that the CXCL14 production was increased in IPFP-MSC compared to the ADSC (subcutaneous adipose tissue-derived MSC) in the early stage of chondrogenesis. FIG. 1A provides the RNA-seq data showing the number of significant genes during MSC chondrogenesis on day 7, day 10, day 14, day 21, and day 28. FIG. 1B provides the Volcano plot of differential expression genes (DEG), wherein the expression of each gene was plotted as the log-fold change of expression ratio for controls; upregulated and downregulated genes were represented by red and blue dots, respectively. FIG. 1C provides the GEO profiles from NCBI showing the kinetics of (CXCL14, IL10, and IL18 gene expressions from human bone marrow stem cells during chondrogenesis. FIG. 1D shows that CXCL14 only can strongly be detected in IPFP-MSC but not in subcutaneous adipose-derived MSC using ELISA (n=3).

FIGS. 2A-2B show that the exogenous recombinant protein CXCL14 increased the chondrogenic potential. FIG. 2A provides the image showing Alcian blue staining analyzed chondrogenic differentiation of pellets with varying ratios of IPFP-MSC. FIG. 2B shows that the exogenous CXCL14 increased the GAG deposition in IPFP-MSC during chondrogenesis on day 3.

FIGS. 3A-3B show that recombinant protein CXCL14 increased cartilage regeneration. FIG. 3A provides the image showing Safranin O staining and IHC staining of collagen II in the tibial articular cartilage of WT mice after ACLT (Scale bars, 100 μm), as well as Safranin O staining and IHC staining of collagen II in mouse joints (n=6) with sham, OA cartilage, and receiving CXCL14; wherein the black arrows indicate reduced cartilage thickness and staining intensity in OA mice. F, femur; T, tibia. FIG. 3B provides the profile of OARSI scores, indicating articular cartilage damage/repair in all groups, wherein the OARSI score of the ACLT group (score of 5.5) was significantly higher than that of the sham group (score of 0.17), and CXCL14 administration improved the score to 2.7 (10 ng) and 1 (20 ng).

FIG. 4 provides the profiles showing recombinant protein CXCL14-induced chondrogenic genes under TGF-b stimulation during chondrogenesis, wherein (CXCL14, SOX9, COL2A1, and ACAN mRNA expressions were measured during chondrocyte differentiation by real-time PCR and showed increased expression.

FIGS. 5A-5B show that recombinant protein CXCL14 triggers the expression of SOSTDC1, an antagonist of BMP2, during chondrogenesis. FIGS. 5A provides the heatmap of 11 significantly upregulated genes identified during chondrogenesis on day 7 following treatment with CXCL14. Genes with changes in expression highlighted in red/blue font are potential candidates. FIGS. 5B provides the validation of the selected significant gene by qPCR, which shows upregulated expression of SOSTDC1 in human ADSCs from non-OA donors and IPFP-MSCs from OA donors. The vehicle group was treated with a medium without containing TGF-beta or other supplements used for chondrogenic induction.

FIGS. 6A-6E show that CXCL14-ADSC spheroid stimulates cartilage function in ACLT-induced OA rats. FIG. 6A provides the immunofluorescence staining, which was performed to detect Collagen type II, SOX9, Aggrecan, and Hoechst 33342 in 3D ADSC spheroids. FIG. 6B provides the Safranin O staining in the tibial articular cartilage of rats after ACLT (Scale bars, 100 μm). Safranin O staining in rat joints (n=6) with sham, OA cartilage, and receiving CXCL14-ADSC-spheroid. Black arrows indicate reduced cartilage thickness and staining intensity in OA mice. FIG. 6C provides the OARSI scores, which reflects the extent of articular cartilage damage and repair, were assessed for all groups. The ACLT group had a significantly higher OARSI score (5.65) compared to the sham group (0.12). Intraarticular administration of CXCL14-ADSC spheroids (1.5×10⁶ cells) improved the OARSI scores, reducing them to 2.06. FIG. 6 D provides the knee swelling result. The knee swelling of OA rats was alleviated after CXCL14-ADSC-spheroid intraarticularly administration. FIG. 6 E shows that the electrical shock supplied by the grid is of constant intensity in the treadmill. The number of electric shocks during treadmill exercise was significantly decreased by intra-articular administration of CXCL14-ADSC-spheroids compared to the OA group treated with PBS.

FIGS. 7A-7G show that CXCL14 peptides can greatly stimulate chondrogenesis. The vehicle group was treated with a medium without containing TGF-beta or other supplements used for chondrogenic induction. FIG. 7A shows the CXCL14 peptides enhanced chondrogenic gene expressions stronger than CXCL14 protein in ADSC. The peptide/protein CXCL14-treated ADSCs and the ('XCL14, SOX9, and COL2A1 genes were analyzed by real-time PCR on day 7. FIG. 7B shows that CXCL14-P enhances the chondrogenic potential of human ADSCs. The chondrogenic progenitor cells were identified using biomarkers such as SOX9, Collagen type II, COMP, and CXCR4. On the third day of differentiation, ADSCs were analyzed by flow cytometry to assess the presence of these specific chondrogenic markers. FIG. 7C shows the analysis of the chondrogenic differentiation of ADSC pellets by Alcian blue staining. The ADSCs treated with CXCL14-P exhibited higher GAG content during the chondrogenesis process. FIG. 7D shows the immunofluorescence staining conducted to detect Collagen type II (green color), SOX9 (red color), Aggrecan (indigo color), and Hoechst 33342 (gray color) in 3D ADSC spheroids treated with CXCL14-P. FIG. 7E shows the cellular uptake of FAM-CXCL14-P in ADSCs observed by using fluorescence microscopy. FIG. 7F shows the Real-time PCR results of one-day treatment with CXCL14-P on day 7 for analyzing the expression of SOX9, COL2A1, COMP, and ACAN genes in CXCL14-P-treated ADSC. FIG. 7G shows the chondrogenic differentiation of pellets with varying ratios of ADSCs assessed by using Alcian blue staining.

FIGS. 8A-8B show that CXCL14-P-ADSC-EV notably enhances the chondrogenesis of ADSCs. FIG. 8A shows the expression of the genes SOX9, COL2A1, ACAN, COMP, SOSTDC1, and ITGB1 induced by CXCL14-P-ADSC-EV. The fold change in gene expression, shown on the y-axis, was calculated relative to the expression of the same genes in untreated CXCL14-P-ADSC-EV. One-way ANOVA was performed. Statistical significance is indicated as follows: *p<0.05, **p<0.01, ***p<0.001, and ****p<0.0001 for EV treatments versus the untreated group. FIG. 8B shows the expressions of Collagen type II, SOX9, COMP, and Hoechst 33342 in 3D ADSC spheroids treated with MSC-EV-CXCL14-P at different time points by analysis of immunofluorescence staining.

### DETAILED DESCRIPTION

Unless otherwise defined, all technical and scientific terms used herein have the same meaning as commonly understood by those skilled in the art to which the present invention pertains. It should be understood that the terminology used herein is for the purpose of describing specific embodiments only, and is not intended to be limiting.

As used herein, the singular forms “a”, “an” and “the” include plural references unless explicitly indicated otherwise. Thus, for example, reference to “a sample” includes a plurality of such samples and their equivalents known to those skilled in the art.

As used herein, the term “Chemokine (C-X-C motif) ligand 14”, “CXCL14 protein” or “CXCL14” refers to a small cytokine belonging to the CXC chemokine family that is also known as BRAK (for breast and kidney-expressed chemokine), which mainly regulates immune cell migration and executes antimicrobial immunity. Yet, the underlying chondrogenic functions and mechanisms are still unknown. In the present invention, one example of CXCL14 used in the present invention is Homo sapiens CXCL14. In the present invention, the term “CSCL14 gene” or “CSCL14” means the gene coding for CXCL14 protein, or the CSCL14 mRNA, such as the Homo sapiens CXCL14 mRNA referring to NCBI Reference Sequence: NM_004887.5.

As used herein, the term “cartilage” includes hyaline cartilage, fibrocartilage, or elastic cartilage, and is not particularly limited. One example of cartilage in the present invention, is knee cartilage.

As used herein, the term “regeneration” refers to an action that, when an organism has lost some of its body or its function, it reforms the tissue or organ of that part to restore it to its original state or restore its function. This regeneration ability is stronger as the system is simple and systematic and the degree of evolution is low.

As used herein, the term “cartilage defect disease” refers to a disease caused by cartilage defects, injuries, or defects caused by cartilage, cartilage tissue and/or joint tissues (synovial membrane, articular capsule, cartilaginous bone, etc.) injured by mechanical stimulation or inflammatory reaction. Such cartilage defect diseases include, but are not limited to, degenerative arthritis, rheumatoid arthritis, fractures, muscle tissue damage, plantar fasciitis, humerus ulcer, calcified myositis, or joint damage caused by fracture nonunion or trauma. In the invention, one particular example is degenerative osteoarthritis.

As used herein, the phrase “therapeutically effective amount” refers to an amount sufficient to treat a disease at a reasonable benefit/risk ratio applicable to medical treatment, and an effective dose level may be determined by the species and severity, age, sex, type of disease, duration of treatment, factors including co-administered drugs, and factors well known in other medical disciplines.

As used herein, the term “subject” refers to the target of administration or implantation, e.g., humans or animals including domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). According to the invention, the subject can be a vertebrate, such as a mammal, a fish, or a bird. In particular examples, the subject may be a human, a non-human primate, a dog, a cat, a horse, a pig, a rabbit, a sheep, a goat, a guinea pig, or a rodent.

The term “patient” as used herein refers to a subject afflicted with one or more diseases or disorders or conditions, such as one or more cartilage defects, who requires medical intervention.

As used herein, the term “treatment” refers to the medical management of a patient with the intent to cure, ameliorate, or stabilize a disease, pathological condition, or disorder (for example, a cartilage defect). This term includes active treatment, that is, treatment directed specifically toward the improvement of a disease, pathological condition, or disorder. In addition, this term includes palliative treatment, that is, treatment designed for the relief of symptoms rather than the curing of the disease, pathological condition, or disorder; and a supportive treatment, which is a treatment employed to supplement another specific therapy directed toward the improvement of the associated disease, pathological condition, or disorder, for example, a therapy for (i) inhibiting the disease, i.e., arresting its development; or (ii) for relieving the disease, i.e., causing regression of the disease.

As used herein, the term “treating” refers to improving or eliminating a disease, pathological condition, or disorder. In the invention, the term “treating” means reducing the effects of a cartilage defect or the symptoms of a cartilage defect.

As used herein, the term “administering” or “administration” refers to any method of providing a disclosed agent or a pharmaceutical preparation comprising a disclosed agent to a subject. Such methods are well known to those skilled in the art and include, but are not limited to: oral administration, transdermal administration, administration by inhalation, nasal administration, topical administration, and parenteral administration, including injectable such as intravenous administration, intra-arterial administration, intramuscular administration, and subcutaneous administration. Administration can be continuous or intermittent. In one particular example of the invention, the composition according to the invention is topically administered to the subject.

The present invention provides a new approach for controlling chondrogenesis and stimulating cartilage repair in a cartilage defect disease, such as degenerative osteoarthritis (OA).

Although the interactions of varieties of growth factors, cytokines, and signaling molecules have been reported to regulate the chondrogenic differentiation of mesenchymal stem cells (MSCs), many signaling factors involved in chondrogenesis but have a risk of inducing fibrotic side effects, for example, transforming growth factor-β (TGF-β). It was unexpectedly found that the CXCL14 gene, which should be potential target for controlling chondrogenesis, as proved by the RNA-sequencing (RNA-seq) of being highly expressed in human bone marrow cells during chondrogenesis. The CXCL14 protein, a novel chemokine, mainly regulates immune cell migration and executes antimicrobial immunity. Yet, the underlying chondrogenic functions and mechanisms are still unknown.

In the present invention, the CXCL14 protein, which has the amino sequence set forth in SEQ ID NO: 1, was found and ascertained to provide an effect to enhance chondrogenesis and correlate with dynamic changes during chondrogenesis. It was demonstrated in the examples that the CXCL14 gene was highly expressed in human bone marrow cells during chondrogenesis in the volcano plot and GEO profiles by RNA-sequencing (RNA-seq). Furthermore, the level of CXCL14 was enhanced during chondrogenesis in infrapatellar fat-pad-derived MSC (IPFP-MSC) from OA patients but not subcutaneous adipose-derived MSC in vitro by Alcian blue assay.

According to the invention, the CXCL14 protein, the peptide fragment thereof may be encapsulated in a stem cell-derived extracellular vesicle (EV) to avoid the degradation.

In the examples of the present invention, it was observed that CXCL14 protein had the function of CXCL14 protein in regulating chondrogenic induction in IPFP-MSC from OA patients in vitro and stimulated cartilage repair in the anterior cruciate ligament transection-induced OA model staining glycosaminoglycan content and type II collagen in vivo. The results suggest that the CXCL14 protein impacts the chondrogenesis inducer and serves as a potential candidate for OA therapy.

In the examples of the present invention, it was also observed that CXCL14-treated ADSCs treatment mitigated cartilage degradation. The knee swelling was reduced following the intra-articular administration of CXCL14-ADSC spheroids. Additionally, the electrical shocks supplied by the grid in the treadmill, which maintain a constant intensity, were used to assess cartilage function. The frequency of electric shocks experienced during treadmill exercise was significantly higher in OA rats but decreased following the intra-articular administration of CXCL14-ADSC spheroids compared to the OA group treated without CXCL14-ADSC spheroids.

In the examples of the present invention, it was found that an CXCL14 peptide (CXCL14-P), which has the sequence set forth in SEQ ID NO: 2, enhances the chondrogenic potential of human ADSCs.

In the examples of the present invention, it was found that CXCL14-P-ADSC-EV (extracellular vesicles of CXCL14-P treated ADSC) significantly boosts the chondrogenesis of ADSCs. The investigation of gene expressions revealed that CXCL14-P-ADSC-EVs significantly enhanced chondrogenic gene expression in ADSCs more effectively than the CXCL14 peptide alone.

The present invention is further illustrated by the following examples, which are provided for the purpose of demonstration rather than limitation.

### EXAMPLES

### Materials and Methods

### Research Ethics and Consent

Thirteen patients with OA aged 50-75 years undergoing joint replacement surgery but without other rheumatic diseases treated with immunosuppressive drugs in the past 3 months were recruited. The patients' infrapatellar fat pad (IPFP) and stem cells (SC) were collected and immediately processed. In addition, all the patients signed consent forms on the using of their IPFP and SC for the experiments. The study had been approved by the Research Ethics Committee of Far Eastern Memorial Hospital, Taipei, Taiwan (111098-F).

### Mice

Wild-type male mice of a C57BL/6 background were bred and maintained under specific pathogen-free conditions in the animal care unit of Far Eastern Memorial Hospital. All of the animal experiments were conducted according to the guidelines of the Association for Assessment and Accreditation of Laboratory Animal Care (AAALAC), and were approved by the Animal Ethics Committee of the Far Eastern Memorial Hospital (IACUC number: IACUC-2022 (4)-MOST-09).

Isolation of MSCs from IPFPs

The IPFP tissue was washed repeatedly with Dulbecco's phosphate-buffered saline (Gibco, cat. no. 21600) containing 5% penicillin-streptomycin and amphotericin B solution (Gibco, cat. no. 15240). The tissue was minced and subjected to enzymatic digestion with 0.1% type I collagenase (Gibco, cat. no. 17100017) diluted in minimum essential medium α (α-MEM, Gibco, cat. no. 120000) and 1% bovine serum albumin (Sigma, cat. no. A7906) for 18 h at 37° C. After digestion, an equal amount of culture medium containing 10% fetal bovine serum was added to stop collagenase activity. The cell solution was sieved through a 100-μm mesh and centrifuged at 500 g for 5 min. The cell pellets were resuspended in culture medium (supplemented with 10% FBS, 1% penicillin-streptomycin, and amphotericin B) and seeded in a 10-cm culture dish. The medium was changed every 72 h until the cells reached 80%-90% confluence.

### Extracellular Vesicles (EVs) Were Isolated and Purified From Adipose Tissue-Derived MSCs

Initially, the cells were cultured in a medium containing serum. After washing the cells three times with PBS, the medium was switched to a serum-free culture medium, and the cells were incubated for 24 hours following cellular nanoporation (CNP). The conditioned medium (CM) was then centrifuged at 300×g for 5 minutes to remove cells and at 2000×g for 20 minutes to eliminate cell debris. The CM was sterilized using a 0.22-μm polyethersulfone (PES) membrane filter. EVs were collected and purified using a tangential flow filtration (TFF) system, equipped with a peristaltic pump (Lefo Science Co., Ltd) and a hollow fiber (MidiKros). The size distribution and concentration of the EV samples were determined by nanoparticle tracking analysis (NTA) using a NanoSight NS300 instrument (Malvern Instruments, MA, US).

### Chondrogenic Differentiation

To obtain a 3D pellet culture, 1×10⁵ MSCs were centrifuged at 1200 rpm for 3 min in polypropylene tubes and cultured for 1 day. To induce chondrogenesis, the pelleted cells were cultured in DMEM-HG (Gibco, cat. no. 121000) containing 1% ITS-Premix (Corning, cat. no. 354352), 1% penicillin streptomycin (Gibco, cat. no. 15240), 1 mM sodium pyruvate (Gibco, cat. no. 11360070), 0.35 mM proline (Sigma-Aldrich, cat. no. P5607), 10 ng/mL TGF-β1 (SinoBio, cat. no. 10804-HNAC), 0.17 mM ascorbic acid 2-phosphate (Sigma-Aldrich, cat. no. A8960), and 100 nM dexamethasone (Sigma-Aldrich, cat. no. D4902). After induction, the chondrogenic differentiation results were evaluated on days 1, 7, 14, and 21.

### ACLT-Induced Knee OA

Surgical ACLT is the most commonly used for generating an animal model of OA. In this study, 8-week-old specific-pathogen-free C57BL/6 mice were subjected to ACLT and medial collateral ligament (MCL) transection for establishing an OA mouse model (n=6). First, the knee joints of the hind limbs were incised along the medial edge of the patellar ligament. After the joint capsule was exposed, the ACL and MCL were transected. After 4 weeks, cartilage destruction and inflammation—signs of OA—were observed in the mice.

### Safranin Olfast Green Staining

After deparaffinization, the sections were stained with hematoxylin for 3 min and differentiated in 1% acid alcohol for 15 s, followed by staining in 0.02% aqueous fast green for 3 min and counterstaining with 0.1% Safranin O for 3 min. Finally, the sections were dehydrated in serial dilutions of ethanol, cleared in xylene, mounted onto glass slides by using neutral gum. Each stained area was observed under a light microscope and photographed using a photomicroscope equipped with a CCD video camera.

### Immunofluorescence Staining

MSC spheroids were fixed with 4% paraformaldehyde (PFA) (PH 7.2) in PBS for 30 minutes at room temperature. The samples were then washed with PBS to remove the PFA. Next, the spheroids were carefully transferred into ice-cold methanol and incubated at −40° C. for 25 minutes. Following fixation, the spheroids were treated with 1 mg/mL sodium borohydride to clean them, and then washed with PBS. After washing, the spheroids were incubated in a blocking solution containing 10% normal rabbit serum and 1x PBS with 0.1% sodium azide for 6 hours or overnight. The samples were then incubated with primary antibodies against Collagen type II (ab34712, Abcam), SOX9 (ab185966, Abcam), COMP (sc-374660, Santa Cruz), and Aggrecan (MA3-16888, Invitrogen) for 24 hours at 4° C. Subsequently, the samples were incubated with Alexa Fluor 488-, Alexa Fluor 555-, or Alexa Fluor 647-conjugated secondary antibodies for 1 hour at room temperature. Fluorescence images were acquired using a Leica SP8 STED confocal microscope.

### Statistical Analysis

All data are presented as mean ± SEM and were analyzed using GraphPad Prism software (Version 6.0). p values were calculated using one-way analysis of variance with a post hoc Bonferroni test with >3 replicates for multiple comparisons and using unpaired Student's t test for two groups.

### Results

### RNA-Seq Reveals CXCL14 was Increased During Human Chondrogenesis

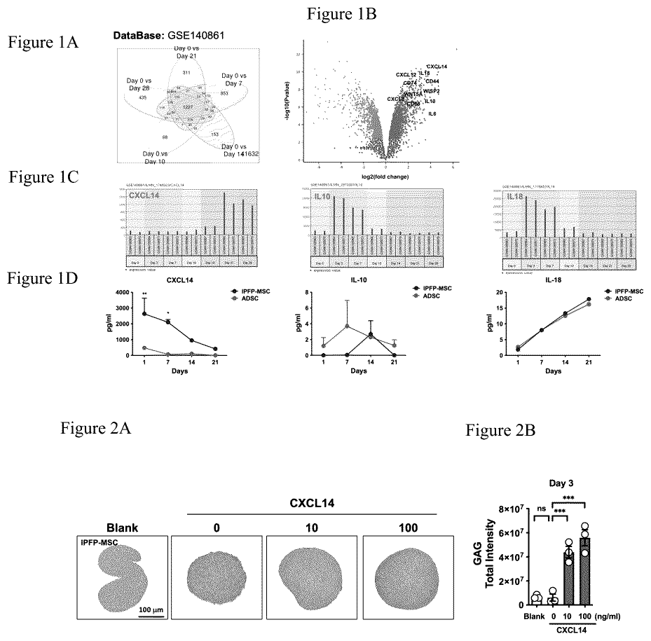

To dissect the kinetic gene expressions and explore the regulatory changes during chondrogenesis, RNA-seq on hBM-MSC was performed. As shown in FIG. 1A, FIG. 1B, and FIG. 1C, RNA-sequencing (RNA-seq) showed the CXCL14, IL10, and IL18 genes, which were highly expressed in human bone marrow cells during chondrogenesis in the volcano plot and GEO profiles; however, only CXCL14 was enhanced during chondrogenesis in infrapatellar fat-pad-derived MSC (IPFP-MSC) from the OA patients in vitro.

As shown in FIG. 1D, CXCL14 only could strongly be detected in IPFP-MSC, which is a higher chondrogenic capacity MSC but not in subcutaneous adipose-derived MSC. It is found in this study that CXCL14 functioned in regulating chondrogenic induction in IPFP-MSC. The results showed that CXCL14 could induce chondrogenesis, suggesting that CXCL14 is able to undergo changes during the chondrogenic induction progress in cartilage.

### Exogenous CXCL14 Stimulated Chondrogenesis and Cartilage Regeneration

CXCL14 was analyzed to determine the stimulation of chondrogenesis and cartilage regeneration. CXCL14 was also analyzed to determine whether CXCL14 can induce chondrogenesis by using IPFP-MSC from OA patients treated with the recombinant protein CXCL14 and investigating the glycosaminoglycan (GAG) content by Alcian blue staining. It was found that CXCL14 functioned in regulating chondrogenic induction in IPFP-MSC from OA patients in vitro. The function of CXCL14 in regulating chondrogenesis was also characterized. As shown in FIG. 2, the exogenous recombinant protein CXCL14 in a dose-dependent stimulated cartilage repair in the anterior cruciate ligament transection-induced OA model (ACLT-induced OA) staining GAG content and type II collagen. FIG. 2A showed the Alcian blue staining analyzed chondrogenic differentiation of pellets with varying ratios of IPFP-MSC, and FIG. 2B showed that the exogenous CXCL14 increased the GAG deposition in IPFP-MSC during chondrogenesis on day 3.

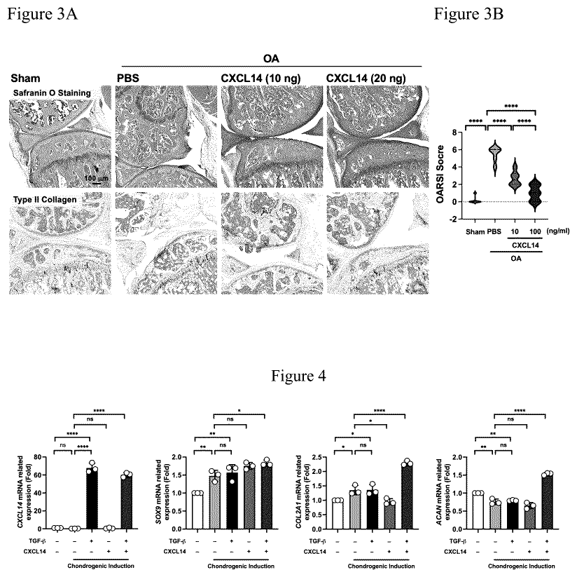

As shown in FIG. 3, CXCL 14 increased cartilage regeneration. FIG. 3A showed the results of the Safranin O staining and IHC staining of collagen II in the tibial articular cartilage of WT mouse joints (n=6) after ACLT (Scale bars, 100 μm) with sham, OA cartilage, and receiving CXCL14. The black arrows indicate reduced cartilage thickness and staining intensity in OA mice. F, femur; T, tibia. FIG. 3B shows that OARSI scores indicated articular cartilage damage/repair in all groups. The OARSI score of the ACLT group (score of 5.5) was significantly higher than that of the sham group (score of 0.17). It was observed that CXCL14 administration could improve the score to 2.7 (10 ng) and 1 (20 ng). Given the above, a remarkable link between CXCL14 and chondrogenesis can be recognized, which suggests the impact of CXCL14 on cartilage repair. In conclusion, the function of CXCL14 in regulating chondrogenesis is characterized.

### CXCL14 Induced Chondrogenic Gene Expression

To better understand the molecular mechanism relating to the control of chondrogenesis modulated by CXCL14, the chondrogenic gene expressions were investigated during chondrogenesis. As shown in FIG. 4, the results reveal that CXCL14 could induce CXCL14, SOX9, COL2A1, and ACAN under a low dose of TGF-β stimulation, suggesting that augmentation of CXCL14 and TGF-β signaling was ascertained for the enhancement of chondrogenesis and cartilage regeneration.

Treatment With CXCL14 Protein LEADS to the upregulation of Potential Candidate Gene, SOSTDC1, BMP2 Antagonist, During MSC Chondrogenesis

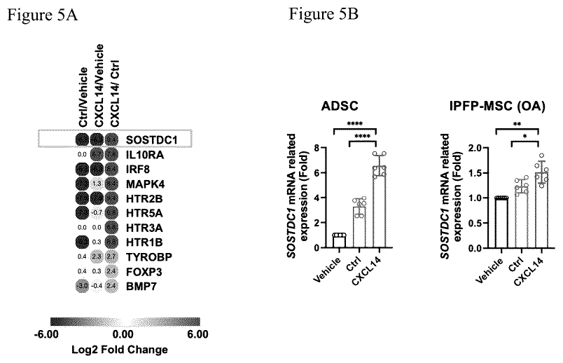

To understand the mechanism of CXCL14 protein treatment during chondrogenesis, we performed RNA-sequencing analysis. RNA-seq results indicated that CXCL14 protein treatment led to the upregulation of several potential candidate genes involved in chondrogenesis. As shown in FIG. 5A, the heatmap displayed 11 significantly upregulated genes on day 7 post-treatment with CXCL14, with changes in expression highlighted in red/blue font to indicate potential candidates. As shown in FIG. 5B, validation of these significant genes via qPCR revealed upregulated expression of SOSTDC1, BMP2 antagonist, in human ADSCs from non-OA donors and IPFP-MSCs from OA donors.

### The CXCL14-ADSC Spheroid Enhances Cartilage Function in ACLT-Induced OA Rats

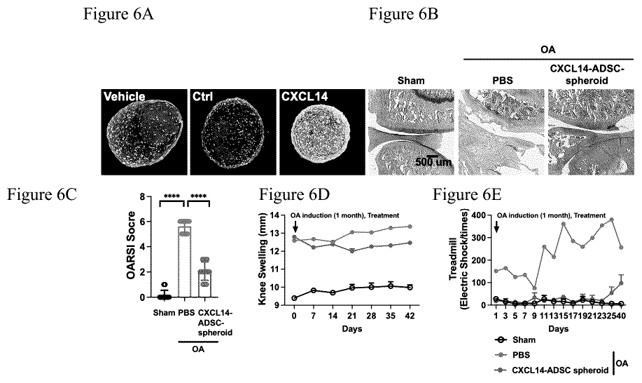

CXCL14-treated ADSCs were employed in a 3D culture. As shown in FIG. 6A, the results indicated that CXCL14 can elevate the expressions of SOX9, Collagen type II, and Aggrecan in ADSC spheroids. Consequently, we administered intra-articular injections of CXCL14-treated ADSC spheroids into the joints of ACLT-induced OA rats. As shown in FIG. 6B, Safranin O staining of the tibial articular cartilage in rats post-ACLT, treated with CXCL14-ADSC spheroids, revealed that CXCL14-ADSC spheroid treatment mitigated cartilage degradation. The ACLT group exhibited a significantly higher OARSI score (5.65) compared to the sham group (0.12). However, as shown in FIG. 6C, intra-articular administration of CXCL14-ADSC spheroids improved the OARSI scores, reducing them to 2.06. As shown in FIG. 6D, knee swelling was reduced following the intra-articular administration of CXCL14-ADSC spheroids. Additionally, the electrical shocks supplied by the grid in the treadmill, which maintain a constant intensity, were used to assess cartilage function. As shown in FIG. 6E, the frequency of electric shocks experienced during treadmill exercise was significantly higher in OA rats but decreased following the intra-articular administration of CXCL14-ADSC spheroids compared to the OA group treated with PBS.

### CXCL14 Peptides Significantly Promote Chondrogenesis

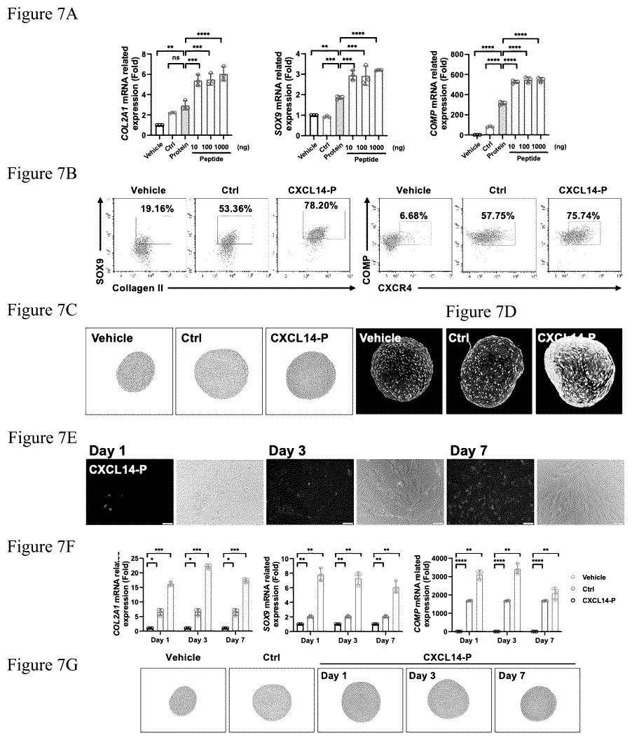

To identify functional peptide sequences within the CXCL14 protein that enhance chondrogenesis, an CXCL14 peptide (CXCL14-P), which has the amino acid sequence set forth in SEQ ID NO: 2, was used for interacting with CXCR4. The synthesized specific CXCL14-P, produced through solid-phase peptide synthesis, was then compared its function to the CXCL14 protein in ADSC chondrogenesis. As shown in FIG. 7A, ADSCs were treated with either the CXCL14-P or protein were analyzed for CXCL14, SOX9, and COL2A1 gene expression using real-time PCR on day 7. According to the result, CXCL14-P treatment resulted in stronger chondrogenic gene expression compared to the protein.

To further verify the presence of chondrogenic proteins in response to CXCL14-P treatment, ADSCs were analyzed during chondrogenesis using biomarkers such as SOX9, Collagen type II, COMP, and CXCR4. On the third day of differentiation, flow cytometry was used to assess these specific chondrogenic markers. As shown in FIG. 7B, the results demonstrated that CXCL14-P enhances the chondrogenic potential of human ADSCs. As shown in FIG. 7C, Alcian blue staining was performed to evaluate the chondrogenic differentiation of ADSC pellets, revealing higher GAG content in CXCL14-P-treated ADSCs. As shown in FIG. 7D, immunofluorescence staining confirmed these findings, showing consistent results for Collagen type II (green), SOX9 (red), Aggrecan (indigo), and Hoechst 33342 (gray) in 3D ADSC spheroids treated with CXCL14-P. To determine the duration of the CXCL14-P chondrogenic function, we replaced the medium containing FAM-CXCL14-P with fresh medium after one day of treatment. ADSCs were monitored until day 7, with cellular uptake of FAM-CXCL14-P observed using fluorescence microscopy, the results were shown in FIG. 7E.

In order to confirm the effect of CXCL14-P on chondrogenesis induction, we administered a one-day treatment followed by a change to fresh medium without CXCL14-P. As shown in FIG. 7F, real-time PCR was conducted on day 7 to analyze the expression of SOX9, COL2A1, COMP, and ACAN genes in CXCL14-P-treated ADSCs. This demonstrated that a one-day treatment with CXCL14-P significantly induced chondrogenic gene expression. Additionally, as shown in FIG. 7G, the chondrogenic differentiation of ADSC pellets with varying ratios was assessed using Alcian blue staining, showing increased GAG deposition on day 7 following CXCL14-P treatment.

### CXCL14-P-ADSC-EV Significantly Boosts the Chondrogenesis of ADSCs

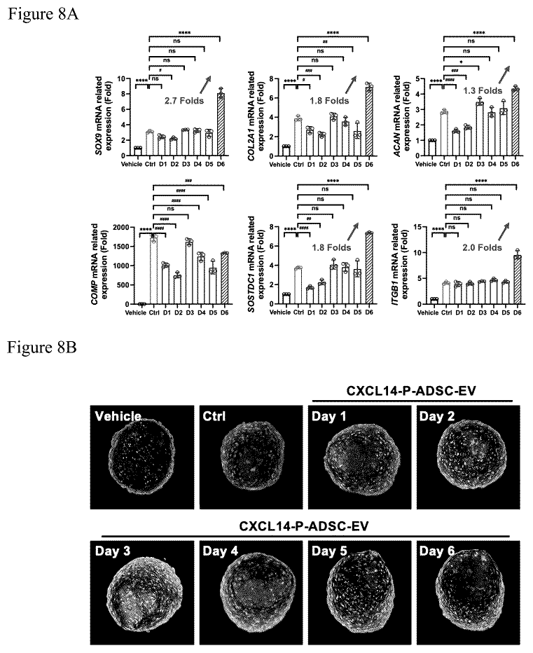

MSC-EVs have been reported to have potential for tissue repair; however, naive EVs lack the enhanced capacity to stimulate chondrogenesis and cartilage repair. Therefore, CXCL14-P was used to stimulate ADSCs to enhance their chondrogenic potential and then extracted the EVs for further evaluation of chondrogenesis. On day 7, ADSCs treated with CXCL14-P-MSC-EVs were analyzed for the expression of SOX9, COL2A1, ACAN, COMP, SOSTDC1, and ITGB1 genes daily from day 1 to day 7 using real-time PCR. As shown in FIG. 8A, the investigation of gene expressions revealed that CXCL14-P-ADSC-EVs significantly enhanced chondrogenic gene expression in ADSCs more effectively than the CXCL14 peptide alone. As shown in FIG. 8B, the SOX9, COMP, and Collagen type II proteins of ADSC spheroids were treated by CXCL14-MSC-EV and demonstrated that significantly increased the expression of proteins related to chondrogenesis.

Given the above, it can be concluded that the recombinant CXCL14 protein and its peptide fragment are linked to chondrogenesis. Moreover, the stem cell-derived extracellular vesicle, which contains the CXCL14 peptide fragment introduced via cellular nanoporation, shows a superior effect in regulating chondrogenesis, indicating its potential as a pharmaceutical composition for treating cartilage regeneration.

While this invention has been described in connection with what is presently considered to be practical exemplary embodiments, it is to be understood that the invention is not limited to the disclosed embodiments. On the contrary, it is intended to cover various modifications and equivalent arrangements included within the spirit and scope of the appended claims.

### References

ADDIN EN.REFLIST 1. E. V. Munsell, D. S. Kurpad, T. A. Freeman, M. O. Sullivan, Histone-targeted gene transfer of bone morphogenetic protein-2 enhances mesenchymal stem cell chondrogenic differentiation. Acta Biomater 71, 156-167 (2018).
2. T. Zhang et al., Cross-talk between TGF-beta/SMAD and integrin signaling pathways in regulating hypertrophy of mesenchymal stem cell chondrogenesis under deferral dynamic compression. Biomaterials 38, 72-85 (2015).
3. Z. Luo et al., Mechano growth factor (MGF) and transforming growth factor (TGF)-beta3 functionalized silk scaffolds enhance articular hyaline cartilage regeneration in rabbit model. Biomaterials 52, 463-475 (2015).
4. H. M. van Beuningen, H. L. Glansbeek, P. M. van der Kraan, W. B. van den Berg, Differential effects of local application of BMP-2 or TGF-beta 1 on both articular cartilage composition and osteophyte formation. Osteoarthritis Cartilage 6, 306-317 (1998).
5. V. Deshmukh et al., Modulation of the Wnt pathway through inhibition of CLK2 and DYRKIA by lorecivivint as a novel, potentially disease-modifying approach for knee osteoarthritis treatment. Osteoarthritis Cartilage 27, 1347-1360 (2019).
6. V. Deshmukh et al., A small-molecule inhibitor of the Wnt pathway (SM04690) as a potential disease modifying agent for the treatment of osteoarthritis of the knee. Osteoarthritis Cartilage 26, 18-27 (2018).
7. K. Gelse et al., Molecular differentiation between osteophytic and articular cartilage—clues for a transient and permanent chondrocyte phenotype. Osteoarthritis Cartilage 20, 162-171 (2012).
8. R. A. Somoza et al., Transcriptome-Wide Analyses of Human Neonatal Articular Cartilage and Human Mesenchymal Stem Cell-Derived Cartilage Provide a New Molecular Target for Evaluating Engineered Cartilage. Tissue Eng Part A 24, 335-350 (2018).
9. C. Dai et al., CXCL14 displays antimicrobial activity against respiratory tract bacteria and contributes to clearance of Streptococcus pneumoniae pulmonary infection. J Immunol 194, 5980-5989 (2015).
10. C. Maerki et al., Potent and broad-spectrum antimicrobial activity of CXCL14 suggests an immediate role in skin infections. J Immunol 182, 507-514 (2009).

What is claimed is:

1. A method for treating a cartilage defect disease in a subject in need thereof, comprising administering a pharmaceutical composition comprising a therapeutically effective amount of CXCL14 protein or peptide fragment thereof, in association with a pharmaceutically acceptable carrier.

2. The method of claim 1, wherein the pharmaceutical composition comprises stem cell-derived extracellular vesicles (EV).

3. The method of claim 2, wherein the EV contains the CXCL14 protein or peptide fragment thereof.

4. The method of claim 1, wherein the CXCL 14 protein is selected from the group consisting of:
(1) a protein comprising an amino acid sequence of SEQ ID NO: 1;
(2) a protein comprising a sequence with at least 80% identity to the amino acid sequence of SEQ ID NO: 1;
(3) a protein comprising a sequence with at least 95% identity to the amino acid sequence of SEQ ID NO: 1; and
(4) a protein comprising a sequence with at least 98% identity to the amino acid sequence of SEQ ID NO: 1.

5. The method of claim 1, wherein the peptide fragment thereof is selected from the group consisting of:
(1) a peptide fragment comprising an amino acid sequence of SEQ ID NO: 2;
(2) a peptide fragment comprising a sequence with at least 80% identity to the amino acid sequence of SEQ ID NO: 2;
(3) a peptide fragment comprising a sequence with at least 95% identity to the amino acid sequence of SEQ ID NO: 2; and
(4) a peptide fragment comprising a sequence with at least 98% identity to the amino acid sequence of SEQ ID NO: 2.

6. The method of claim 4, wherein the CXCL14 protein is the amino acid sequence of SEQ ID NO: 1.

7. The method of claim 5, wherein the peptide fragment thereof is the amino acid sequence of SEQ ID NO: 2.

8. The method of claim 1, which further comprises a cell therapy or stem cell-based therapy.

9. The method of claim 1, wherein the cartilage defect disease is degenerative osteoarthritis (OA).

10. The method of claim 2, wherein the stem cell-derived extracellular vesicle (EV) is derived from mesenchymal stem cell (MSC) or adipose-derived stem cell (ADSC).

11. The method of claim 1, wherein the CXCL14 protein or the peptide fragment thereof is administered to the cartilage damage of the subject via an injection or a surgery.

12. The method of claim 3, wherein the EV containing the CXCL14 protein or the peptide fragment thereof is administered to the cartilage damage of the subject via an injection or a surgery.

13. A pharmaceutical composition for treating the cartilage defect disease, comprising a therapeutically an effective amount of CXCL14 protein or peptide fragment thereof, in association with a pharmaceutically acceptable carrier.

14. The pharmaceutical composition of claim 13, which further comprises stem cell-derived extracellular vesicles (EV).

15. The pharmaceutical composition of claim 14, wherein the EV contains the CXCL14 protein or peptide fragment thereof.

16. The pharmaceutical composition of claim 13, wherein the CXCL14 protein is selected from the group consisting of:
(1) a protein comprising an amino acid sequence of SEQ ID NO: 1;
(2) a protein comprising a sequence with at least 80% identity to the amino acid sequence of SEQ ID NO: 1;
(3) a protein comprising a sequence with at least 95% identity to the amino acid sequence of SEQ ID NO: 1; and
(4) a protein comprising a sequence with at least 98% identity to the amino acid sequence of SEQ ID NO: 1.

17. The pharmaceutical composition of claim 13, wherein the peptide fragment thereof is selected from the group consisting of:
(1) a peptide fragment comprising an amino acid sequence of SEQ ID NO: 2;
(2) a peptide fragment comprising a sequence with at least 80% identity to the amino acid sequence of SEQ ID NO: 2;
(3) a peptide fragment comprising a sequence with at least 95% identity to the amino acid sequence of SEQ ID NO: 2; and
(4) a peptide fragment comprising a sequence with at least 98% identity to the amino acid sequence of SEQ ID NO: 2.

18. The pharmaceutical composition of claim 16, wherein the CXCL14 protein is the amino acid sequence of SEQ ID NO: 1.

19. The pharmaceutical composition of claim 17, wherein the peptide fragment thereof is the amino acid sequence of SEQ ID NO: 2.

20. The pharmaceutical composition of claim 14, wherein the stem cell-derived extracellular vesicle (EV) is derived from mesenchymal stem cell (MSC) or adipose-derived stem cell (ADSC).


SEQUENCE LISTING (2 sequences)

SEQ ID NO: 1 - protein, 111 aa, Homo sapiens
MSLLPRRAPPVSMRLLAAALLLLLLALYTARVDGSKCKCSRKGPKIRYSDVKKLEMKPKYPHCEEKMVIITTKSVSRYRGQEHCLHPKLQSTKRFIKWYNAWNEKRRVYEE

SEQ ID NO: 2 - protein, 14 aa, Homo sapiens
ITTKSVSRYRGQEH